(19) United States
(12) Patent Application Publication
(10) Pub. No.: US 2015/0353864 A1
(43) Pub. Date: Dec. 10, 2015

(54) Process for the Production of Bio-Naphtha from Complex Mixtures of Natural Occurring Fats & Oils

(71) Applicant: TOTAL RESEARCH & TECHNOLOGY FELUY, SENEFFE (BE)

(72) Inventors: Walter VERMEIREN, Houthalen (BE); Chantal DIVERCHY, Bousval (BE)

(21) Appl. No.: 14/761,646

(22) PCT Filed: Jan. 21, 2014
(86) PCT No.: PCT/EP2014/051152

§ 371 Date: Jul. 17, 2015

(30) Foreign Application Priority Data
Jan. 21, 2013 (EP) 13152095.9

(51) Int. Cl. C11B 3/00; C07C 1/207; C10G 3/00; C07C 4/04; C11C 3/00; C10L 3/12; C11B 3/06; C11B 3/14; C11C 1/02; C11C 1/10; C10L 1/02; C10L 1/04

(52) U.S. Cl. CPC C11B 3/001; C10L 1/026; C07C 1/2078; C10G 3/00; C10G 3/46; C07C 4/04; C10L 1/04; C10L 3/12; C11B 3/006; C11B 3/06; C11B 3/14; C11C 1/025; C11C 1/10; C11C 3/003; C10L 2200/0476; C10L 2290/543; C07C 2523/883; C07C 2521/04; C10L 2200/0469

(57) ABSTRACT

A process for making a bio-diesel and a bio-naphtha from a complex mixture of natural occurring fats & oils may include subjecting the complex mixture to a refining treatment for removing non-triglyceride and non-fatty acid components, thereby obtaining refined oils. The complex mixture or refined oils may be subjected hydrolysis for obtaining glycerol and free fatty acids. The free fatty acids may be subjected to fractionation for obtaining: a liquid part (phase L); and a solid part (phase S). The phase L may be transformed into alkyl-esters as bio-diesel by an esterification. The phase S may be transformed into linear paraffins as the bio-naphtha by: hydrodeoxygenation or decarboxylation of the free fatty acids; or obtaining fatty acids soaps from the phase S and decarboxylation of the fatty acids soaps.

### FIELD OF THE INVENTION

The present invention relates to the production of bio-naphtha and bio-distillates in an integrated bio-refinery from complex mixtures of natural occurring fats & oils. The limited supply and increasing cost of crude oil and the need to reduce emission of fossil based carbon dioxides has prompted the search for alternative processes for producing hydrocarbon products such as bio-naphtha and bio-diesel. The bio-naphtha can be used as feedstock of conventional steamcracking. Made up of organic matter from living organisms, biomass is the world's leading renewable energy source.

In the following, “bio-diesel” is sometimes referred to as “bio-distillates”

### BACKGROUND OF THE INVENTION

Made from renewable sources, bio-distillates as an alternative fuel for diesel engines is becoming increasingly important. In addition to meeting engine performance and emissions criteria/specifications, bio-distillates has to compete economically with petroleum-distillates and should not compete with food applications for the same triglycerides. Vegetable oils partially or fully refined and of edible-grade quality, are currently predominant feedstock for bio-diesel production. The prices of these oils are relatively high for fuel-grade commodities.

These considerations have led to efforts to identify less expensive materials that could serve as feedstock for bio-diesel production and to design chemical processes for their conversion. Thus, animal fats have been converted to bio-diesel [C. L. Peterson, D. L. Reece, B. L. Hammond, J. Thompson, S. M. Beck, “processing, characterization and performance of eight fuels from lipids”, Applied Engineering in Agriculture. Vol. 13(1), 71-79, 1997; F. Ma, L.D. Clements and M. A. Hanna, “The effect of catalyst, free fatty acids and water on transesterification of beef tallow”, Trans ASAE 41 (5) (1998), pp. 1261-1264], and substantial efforts have been devoted to the development of waste restaurant grease [M. Canakci and J. Van Gerpen, “Bio-destillates production from oils and fats with high free fatty acids”, Trans. ASAE 44 (2001), pp. 1429-1436; Y. Zhang, M. A. Dube, D. D. McLean and M. Kates, “Bio-destillates production from waste cooking oil. 1. Process design and technological assessment”, Bioresour. Technol. 89 (2003), pp. 1-16; W.-H. Wu, T. A. Foglia, W. N. Marmer, R. O. Dunn, C.E. Goering and T. E. Briggs, J. Am. Oil Chem. Soc. 75 (1998) (9), p. 1173], largely the spent product of the deep fat frying of foods, as a bio-diesel feedstock.

The industrial chemistry of fats & oils is a mature technology, with decades of experience and continuous improvements over current practices. Natural fats & oils consist mainly of triglycerides and to some extent of free fatty acids (FFA). Many different types of triglycerides are produced in nature, either from vegetable as from animal origin. Fatty acids in fats & oils are found esterified to glycerol (triacylglycerol). The acyl-group is a long-chain (C₁₂-C₂₂) hydrocarbon with a carboxyl-group at the end that is generally esterified with glycerol. Fats & oils are characterized by the chemical composition and structure of its fatty acid moiety. The fatty acid moiety can be saturated or contain one or more double bonds. Bulk properties of fats & oils are often specified as “saponification number”, “Iodine Value”, “unsaponification number”. The “saponification number”, which is expressed as grams of fat saponified by one mole of potassium hydroxide, is an indication of the average molecular weight and hence chain length. The “Iodine value”, which is expressed as the weight percent of iodine consumed by the fat in a reaction with iodine monochloride, is an index of unsaturation.

Some typical sources of fats & oils and respective composition in fatty acids are given by way of example in Table 1.

TABLE 1

| | | Cotton- | | | Palm | | | | Rice | Rape- |
|---|---|---|---|---|---|---|---|---|---|---|
| | Symbol | seed | Coconut | Corn | Kernel | Peanut | Palm | Linseed | brawn | seed |
| Saturated | | | | | | | | | | |
| Caproic | 6:0 | | 0.4 | | 0.2 | | | | | |
| Caprylic | 8:0 | | 7.3 | | 3.3 | | | | | |
| Capric | 10:0 | | 6.6 | | 3.5 | | | | | |
| Lauric | 12:0 | | 47.8 | | 47.8 | | 0.2 | | | |
| Myristic | 14:0 | 0.9 | 18.1 | | 16.3 | 0.1 | 1.1 | | 0.4 | |
| Palmitic | 16:0 | 24.7 | 8.9 | 10.9 | 8.5 | 11.6 | 44.1 | 6.0 | 19.8 | 3.9 |
| Margaric | 17:0 | | | | | | | | | |
| Stearic | 18:0 | 2.3 | 2.7 | 1.8 | 2.4 | 3.1 | 4.4 | 2.5 | 1.9 | 1.9 |
| Arachidic | 20:0 | 0.1 | 0.1 | | | 1.5 | 0.2 | 0.5 | 0.9 | 0.6 |
| Behenic | 22:0 | | | | | 3.0 | | | 0.3 | 0.2 |
| Lignoceric | 24:0 | | | | | 1.0 | | | | 0.2 |
| TOTAL | | 28.0 | 91.9 | 22.7 | 82.0 | 20.3 | 50 | 9.0 | 23.3 | 6.8 |
| Unsaturated | | | | | | | | | | |
| Myristoleic | 14:1 w-5 | | | | | | | | | |
| Palmitoleic | 16:1 w-7 | 0.7 | | | | 0.5 | | 0.1 | 0.2 | |
| Heptadecenoic | 17:1 w-15 | | | | | | | | | |
| Oleic | 18:1 w-9 | 17.6 | 6.4 | 24.2 | 15.4 | 38.0 | 37.5 | 19.0 | 42.3 | 64.1 |
| Linoleic | 18:2 w-6 | 53.3 | 1.6 | 58.0 | 2.4 | 41.0 | 10 | 24.1 | 31.9 | 18.7 |
| Linolenic | 18:3 w-3 | 0.3 | | 0.7 | | | | 47.4 | 1.2 | 9.2 |
| Gadolenic | 20:1 w-9 | | | | | 1.0 | 0.5 | | 0.5 | 1.0 |
| TOTAL | | 72.0 | 8.1 | 77.3 | 18.0 | 79.7 | 50 | 91 | 76.7 | 93.2 |
| Polyunsaturated | | | | | | | | | | |
| Ricinoleic | 18 | | | | | | | | | |
| Rosin acids | — | | | | | | | | | |
| % FFA | | 0.5-0.6 | 1.0-3.5 | 1.7 | 0.1 | 0.8 | 2-14 | 2 | 5-15 | 0.5-3.8 |

| | | | Soy- | Sun- | | | Butter | Tallow | | | |
|---|---|---|---|---|---|---|---|---|---|---|---|
| | Symbol | Olive | bean | flower | Linola | Lard | fat | Grease | Tall | Castor | Jatropha |
| Saturated | | | | | | | | | | | |
| Caproic | 6:0 | | | | | | 2 | | | | |
| Caprylic | 8:0 | | | | | | 2 | | | | |
| Capric | 10:0 | | | | | | 3 | | | | |
| Lauric | 12:0 | | | 0.5 | | 0.5 | 3.5 | | | | |
| Myristic | 14:0 | 0.02 | 0.1 | 0.2 | | 1.5 | 11 | 3 | | | |
| Palmitic | 16:0 | 10.5 | 11.0 | 6.8 | 5.6 | 26 | 26 | 26 | 2 | 1.0 | 14.6 |
| Margaric | 17:0 | 0.05 | | | | 0.5 | | 0.5 | | | |
| Stearic | 18:0 | 2.6 | 4.0 | 4.7 | 4.0 | 13.5 | 11 | 22.5 | 1 | 1.0 | 7.4 |
| Arachidic | 20:0 | 0.4 | 0.3 | 0.4 | | | 2 | | 0.5 | | |
| Behenic | 22:0 | 0.2 | 0.1 | | | | | | | | |
| Lignoceric | 24:0 | 0.1 | | | | | | | | | |
| TOTAL | | 13.87 | 15.5 | 12.6 | 9.6 | 42.0 | 60.5 | 52.0 | 3.5 | 2.0 | 22.0 |
| Unsaturated | | | | | | | | | | | |
| Myristoleic | 14:1 w-5 | | | | | | | 0.5 | | | |
| Palmitoleic | 16:1 w-7 | 0.6 | 0.1 | 0.1 | | 4 | 2 | 2.5 | | | 0.8 |
| Heptadecenoic | 17:1 w-15 | 0.09 | | | | 0.5 | 3 | 0.5 | | | |
| Oleic | 18:1 w-9 | 76.9 | 23.4 | 18.6 | 15.9 | 43 | 26 | 43 | 16 | 3.0 | 47.5 |
| Linoleic | 18:2 w-6 | 7.5 | 53.2 | 68.2 | 71.8 | 9 | 2.5 | 1.5 | 20 | 4.2 | 28.7 |
| Linolenic | 18:3 w-3 | 0.6 | 7.8 | 0.5 | 2.0 | 0.5 | 4 | | | 0.3 | 1.0 |
| Gadolenic | 20:1 w-9 | 0.3 | | | | 1 | | | 0.5 | | |
| TOTAL | | 86.13 | 84.5 | 87.4 | 90.4 | 58.0 | 37.5 | 48.0 | 54.5 | 7.5 | 78.0 |
| Polyunsaturated | | | | | | | 2 | | 4 | | |
| Ricinoleic | 18 | | | | | | | | | 89.5 | |
| Rosin acids | — | | | | | | | | 40 | | |
| % FFA | | 0.5-3.3 | 0.3-1.6 | 0.1-1.5 | 0.3 | 0.5 | | 5-20 | | | |

Bio-distillates feedstock are classified based on their free fatty acid (FFA) content as follows [J. A. Kinast, “Production of bio-distillates from multiple feedstock and properties of bio-distillates and bio-distillates/-distillates blends”, NREL/SR-510-31460 report (2003)1:
Refined oils, such as soybean or refined canola oils (FFA<1.5%);
Low free fatty acid yellow greases and animal fats (FFA<4.0%);
High free fatty acid greases and animal fats (FFA>20.0%).

Bio-diesel is currently produced by transesterification of triglyceride with methanol, producing methyl-ester and glycerol. This transesterification is catalyzed by homogeneous or heterogeneous basic catalyst. Typically homogeneous catalyst are alkali hydroxides or alkali alkoxides and typical heterogeneous catalyst are alkaline earth or zinc oxide materials, like zinc or magnesium-aluminate spinels. The presence of free fatty acids (FFA) in the raw triglycerides is a cumbersome for the production of bio-diesel as the FFA's react stoechiometrically with the basic catalyst producing alkali or alkaline soaps. This means that fats & oils that contain significant amounts of FFA's cannot be employed directly for bio-diesel production with this process. Several technical solutions have been proposed: (i) starting with an acid catalyzed interesterification with additional glycerol to convert FFA's into glycerides prior to the basic transesterification; (ii) prior to the basic catalyzed transesterification the FFA's are removed by steam and/or vacuum distillation. The latter results in a net loss of feedstock for the production of bio-diesel. Eventually, the so produced FFA's can be converted by acid catalysis into esters in a separate process unit. FFA's can be present in triglycerides in different concentrations and can be present as such resulting from the extraction process or can be produced during storage as of the presence of trace amounts of lipase enzyme that catalyze the triglyceride hydrolyzis or can be produced during processing, like thermal treatments during cooking.

There are other potential feedstock available at this time, namely trap and sewage grease and other very high free fatty acid greases who's FFA can exceed 50%.

The main sources of fats & oils are palm and palm kernels, soybeans, rapeseed, sunflower, coconut, corn, animal fats, milk fats.

Potentially new sources of triglycerides will become available in the near future, namely those extracted from Jatropha and those produced by microalgues. These microalgues can accumulate more then 30 wt % of lipids on dry basis and they can either be cultivated in open basin, using atmospheric CO₂ or in closed photobioreactors. In the latter case, the required CO₂ can originate from the use of fossil hydrocarbons that are captured and injected into the photobioreactor. Main sources of fossil CO₂ are power stations, boilers used in refineries and steamcrackers furnaces used to bring hydrocarbon streams at high temperature or to supply heat of reactions in hydrocarbon transformations in refineries and steamcrackers. In particular steamcracking furnaces produce a lot of CO₂. In order to enhance the CO₂ concentration in flue gases of these furnaces, techniques like oxycombustion, chemical looping or absorption of CO₂ can be employed. In oxycombustion, oxygen is extracted from air and this pure oxygen is used to burn hydrocarbon fuels as to obtain a stream only containing water and CO₂, allowing concentrating easily the CO₂ for storage or re-utilization. In chemical looping, a solid material acts as oxygen-transfer agent from a re-oxidation zone where the reduced solid is re-oxidized with air into oxidized solid to a combustion zone, where the hydrocarbon fuel is burned with the oxidized solid and hence the effluent resulting from the combustion zone only contains water and CO₂. Absorption of CO₂ can be done with the help of a lean solvent that has a high preferential to absorb CO₂ under pressure and typically at low temperature and will release the CO₂ when depressurised and/or heated. Rectisol® and Selexol® are commercial available technologies to remove and concentrate CO₂. Other sources of CO₂ are the byproduct from carbohydrates fermentation into ethanol or other alcohols and the removal of excess CO₂ from synthesis gas made from biomass or coal gasification.

US 2007/0175795 reports the contacting of a hydrocarbon and a triglyceride to form a mixture and contacting the mixture with a hydrotreating catalyst in a fixed bed reactor under conditions sufficient to produce a reaction product containing diesel boiling range hydrocarbons. The example demonstrates that the hydrotreatment of such mixture increases the cloud point and pour point of the resulting hydrocarbon mixture.

US 2004/0230085 reports a process for producing a hydrocarbon component of biological origin, characterized in that the process comprises at least two steps, the first one of which is a hydrodeoxygenation step and the second one is an isomerisation step. The resulting products have low solidification points and high cetane number and can be used as diesel or as solvent.

US 2007/0135669 reports the manufacture of branched saturated hydrocarbons, characterized in that a feedstock comprising unsaturated fatty acids or fatty acids esters with C1-C5 alcohols, or mixture thereof, is subjected to a skeletal isomerisation step followed by a deoxygenation step. The results demonstrate that very good cloud points can be obtained.

US 2007/0039240 reports on a process for cracking tallow into diesel fuel comprising: thermally cracking the tallow in a cracking vessel at a temperature of 260-371° C., at ambient pressure and in the absence of a catalyst to yield in part cracked hydrocarbons.

U.S. Pat. No. 4,554,397 reports on a process for manufacturing olefins, comprising contacting a carboxylic acid or a carboxylic ester with a catalyst at a temperature of 200-400° C., wherein the catalyst simultaneously contains nickel and at least one metal from the group consisting of tin, germanium and lead.

It has been discovered a process to make bio-naphtha and bio-diesel in an integrated biorefinery from all kinds of natural triglycerides or fatty acids. In said process crude fats & oils are optionally refined, either physically or chemically, to remove all non-triglyceride and non-fatty acid components. The complex mixture or the refined oils are next subjected to an hydrolysis step for obtaining free fatty acids and glycerol. The free fatty acids are then fractionated in both liquid and solid fractions. This process aims at separating a starting material into a low melting fraction, the liquid fraction, consisting of free fatty acids having double bonds in the acyl-moieties and a high melting fraction, the solid fraction, consisting of free fatty acids having saturated or substantially saturated acyl-moieties. This process allows optimising the use of the different molecules constituting the natural fats & oils. Bio-destillates require specific cold-flow properties that requires double bonds in the acyl-moiety. On the other hand, the quality of a steamcracker feedstock is better when the hydrocarbon is saturated and linear.

The liquid fraction, potentially mixed with some limited solid fraction, is esterified with a C₁ to C₅ monofunctional alcohol to produce alkyl fatty esters, called also bio-diesel. The amount of solid fraction should be so that the final cold-flow properties are according to the local market specifications.

The solid fraction, potentially mixed with some liquid fraction, can be converted to produce bio-naphtha and optionally bio-propane. The solid fraction can be hydrodeoxygenated or decarboxylated to bio-naphtha. The solid fraction can also be saponified to produce fatty acid soaps that can subsequently be decarboxylated.

As several sources of fats & oils are not suitable to be converted in ester-type bio-diesel because they contain too much saturated acyl-moieties that result in high pour-points and hence improper cold-flow properties, the present invention solves this problem by an appropriate separation of the free fatty acids issued from the starting complex mixtures, allowing an optimal usage of fats & oils for making bio-diesel and bio-naphtha.

The use of a biofeed is a possible solution in the search of alternative raw material for the naphthacracker. Nevertheless, using this type of feed can lead to corrosion problems and excessive fouling because of oxygenates forming from the oxygen atoms in the biofeed. Also existing steamcrackers are not designed to remove high amounts of carbonoxides that would result from the steamcracking of these biofeedstock. According to the present invention, such a problem can be solved by hydrodeoxygenating/decarboxylating (or decarbonylating) this biofeed before its injection into the steam cracker. Thanks to this hydrodeoxygenation/decarboxylation (or decarbonylation), the negative effect due to the production of CO and CO₂ and traces of low molecular weight oxygenates (aldehydes and acids) in the steam cracker is reduced.

Another advantage is of course the production of bio-monomers in the steam cracker.

### BRIEF DESCRIPTION OF THE INVENTION

The subject-matter of the present invention is a process for making a bio-diesel and a bio-naphtha from a complex mixture of natural occurring fats & oils, wherein
said complex mixture is optionally subjected to a refining treatment for removing the major part of the non-triglyceride and non-fatty acid components, thereby obtaining refined oils;
said complex mixture or refined oils are subjected to a hydrolysis step for obtaining glycerol and a mixture of free fatty acids;
said mixture of free fatty acids is subjected to a fractionation step by fractional crystallization for obtaining:
   a liquid or substantially liquid free fatty acids part (phase L); and
   a solid or substantially solid free fatty acids part (phase S); and
said phase L is transformed into alkyl-esters as bio-diesel by an esterification;
said phase S is transformed into linear or substantially linear paraffins as the bio-naphtha :
   by hydrodeoxygenation or decarboxylation of the free fatty acids
   or from said phase S are obtained fatty acids soaps that are transformed into linear or substantially linear paraffins as the bio-naphtha by decarboxylation of the soaps.

By “bio-naphtha”, we mean naphtha produced from renewable sources by hydrotreatment of these renewable sources. It is a hydrocarbon composition, consisting of mainly paraffins and that can be used for the steamcracking to produce light olefins, dienes and aromatics. The molecular weight of this bio-naphtha ranges from hydrocarbons having 8 to 24 carbons, preferably from 10 to 18 carbons.

By “substantially linear paraffins”, we mean a composition of paraffins consisting of at least 90% by weight of linear paraffins.

Said complex mixture of natural occurring fats & oils can be selected among vegetable oils and animal fats, preferentially inedible highly saturated oils, waste food oils, by-products of the refining of vegetable oils, and mixtures thereof. Specific examples of these fats & oils have been previously mentioned in the present specification.

Said refined fats & oils can be advantageously subjected to a hydrolysis step for obtaining free fatty acids and glycerol. Said mixture of free fatty acids can be advantageously fractioned into said phases L and S by a fractional crystallization method which consists in a controlled cooling down during which the free fatty acids of said complex mixture with saturated or substantially saturated acyl-moieties crystallize and precipitate from the mixture forming said phase S, while the free fatty acids with unsaturated or substantially unsaturated acyl-moieties remain liquid forming said phase L, both phases being then separated by simple filtration or decantation or centrifugation.

By “substantially saturated acyl-moieties”, we refer to a composition of saturated fatty acids consisting of at least 90% by weight of saturated fatty acids.

By “substantially unsaturated acyl-moieties”, we refer to a composition of unsaturated fatty acids consisting of at least 50% by weight, preferably 75% by weight, of unsaturated fatty acids.

Furthermore, said fractional crystallization method can be conducted in the absence of solvent.

Said phase L can be esterified with a C₁-C₅ monofunctional alcohol in order to produce alkyl fatty esters as bio-diesel. Said alcohol can be methanol.

Said fatty acid soaps can be obtained by neutralization of free fatty acids, obtained from hydrolysis of the fats & oils.

Said phase S can be transformed into linear or substantially linear paraffins as bio-naphtha by hydrodeoxygenation or decarboxylation or decarbonylation of the fatty acids, said hydrodeoxygenation or decarboxylation or decarbonylation being conducted in the presence of hydrogen and of at least one catalyst. The catalyst(s) can be selected among Ni, Mo, Co or mixtures like NiW, NiMo, CoMo, NiCoW, NiCoMo, NiMoW and CoMoW oxides or sulphides as catalytic phase, preferably supported on high surface area carbon, alumina, silica, titania or zirconia or Group 10 (Ni, Pt or Pd) or Group 11 (Cu or Ag) metals or alloy mixtures supported on high surface area carbon, magnesia, zinc-oxide, spinels (Mg₂Al₂O₄, ZnAl₂O₄), perovskites (BaTiO₃, ZnTiO₃), calciumsilicates (like xonotlite), alumina, silica or silica-alumina's or mixtures of the latter. It is preferred that the support for the catalytic active phase exhibit low acidity, preferable neutral or basic in order to avoid hydro-isomerisation reactions that would result in branched paraffins and cracking. The hydrolysis (splitting) can be carried out in presence steam thermally at 15 to 75 bars and at 50-300° C. or catalytically, for example with basic catalysts, like MgO, CaO, ZnO, spinels (Mg2Al2O4, ZnAl2O4), perovskites (BaTiO3, ZnTiO3), calciumsilicates (like xonotlite) or basic alumina or with acidic catalysts, like sulphuric acid. Detailed information about fat & oil splitting has been published by Sonntag (Sonntag, N., J. Am. Oil. Chem. Soc., 56, p. 729, 1979 and Bailey's Industrial Oil and Fat Products, ed. F. Shahidi, 2005, John Wiley & Sons). In the Colgate-Emery process, heated liquid lipid is introduced at the bottom of a vertical tubular reactor. Heated water enters at the top. As the fats & oils rises through the descending water under pressure, a continuous zone of high water solubility in oil establishes, wherein hydrolysis occurs. Effluent from the column is recovered, fatty acids from one outlet and an aqueous glycerol stream from the other. The presence of small amounts of mineral acids, such as sulfuric acid or sulfonic acids or certain metal oxides, such as zinc or magnesium oxide, accelerates the splitting reaction. These metal oxides are true catalysts and they assist also in the formation of emulsions.

Said phase S can be transformed into linear or substantially linear paraffins as bio-naphtha by decarboxylation of the free fatty acids on basic oxides, like alkaline oxides, alkaline earth oxides, lanthanide oxides, zinc-oxide, spinels (Mg₂Al₂O₄, ZnAl₂O₄), perovskites (BaTiO₂, ZnTiO₃), calciumsilicates (like xonotlite), either as bulk material or dispersed on neutral or basic carriers, on basic zeolites (like alkali or alkaline earth low silica/alumina zeolites obtained by exchange or impregnation).

The hydrodeoxygenation of the free fatty acids can be carried out at a temperature from 200 to 500° C., preferably from 280 to 400° C., under a pressure from 1 MPa to 10 MPa (10 to 100 bars), for example of 6 MPa, and with a hydrogen to feedstock ratio from 100 to 2000N1/1, for example of 600 N1 H₂/l oil. The decarboxylation of the free fatty acids can be carried out at 100 to 550° C. in absence or presence of hydrogen at pressures ranging from 0.01 up to 10 MPa (0.1 to 100 bars). The hydrogen to feedstock ratio can be from 0 to 2000 N1/1.

Said phase S can also be transformed into linear or substantially linear paraffins as bio-naphtha by thermal decarboxylation of fatty acid soaps. These soaps are obtained by neutralization of phase S fatty acids obtained by hydrolysis of refined fats & oils and splitting. A soap is a metal salt of the corresponding fatty acid.

The present invention also relates to the use of the bio-naphtha as obtained in the above mentioned process, as a direct feedstock of a steamcracker, said bio-naphtha being used as such, or together with the bio-propane when produced by the above-mentioned process, or as blended with at least a conventional feedstock selected among LPG, naphtha and gasoil, in order to obtain cracked products including bio-ethylene, bio-propylene, bio-butadiene, bio-isoprene, bio-(di)cyclopentadiene, bio-piperylenes, bio-benzene, bio-toluene, bio-xylene and bio-gasoline.

According the above-described use, said feedstock can be mixed with steam in a ratio of 0.2 to 1.0 kg steam per kg feedstock, preferentially of 0.3 to 0.5 kg steam per kg feedstock and the mixture is heated up to a temperature of 750-950° C. at a residence time of 0.05 to 0.5 seconds.

The above-described use may be for steamcracking such as to obtain an ethylene to methane weight ratio, resulting from the cracking of bio-naphtha, of at least 3.

Moreover, the present invention relates to a process for steam cracking a feedstock as defined above, wherein said feedstock is mixed with steam, having a steam/feedstock ratio of at least 0.2 kg per kg of feedstock. This mixture is sent through the heated coils, having a coil outlet temperature of at least 700° C. and a coil outlet pressure of at least 1.2 bara.

### DETAILED DESCRIPTION OF THE INVENTION

All crude fats & oils obtained after rendering, crushing or solvent extraction inevitably contain variable amounts of non-triglyceride components such as free fatty acids, mono and diglycerides, phosphatides, sterols, tocopherols, tocotrienols hydrocarbons, pigments (gossypol, chlorophyll), vitamins (carotenoids), sterols glucosides, glycolipids, protein fragments, traces of pesticides and traces metals, as well as resinous and mucilaginous materials. The quantities of the non-glycerides vary with the oil source, extraction process, season and geographical source. Removal of the non-triglyceride components, which interfere with further processing and cause the oil to darken, foam, smoke, precipitate and develop off-flavours, is the objective of the refining process.

Refining Pretreatment

Choice of the Refining Method

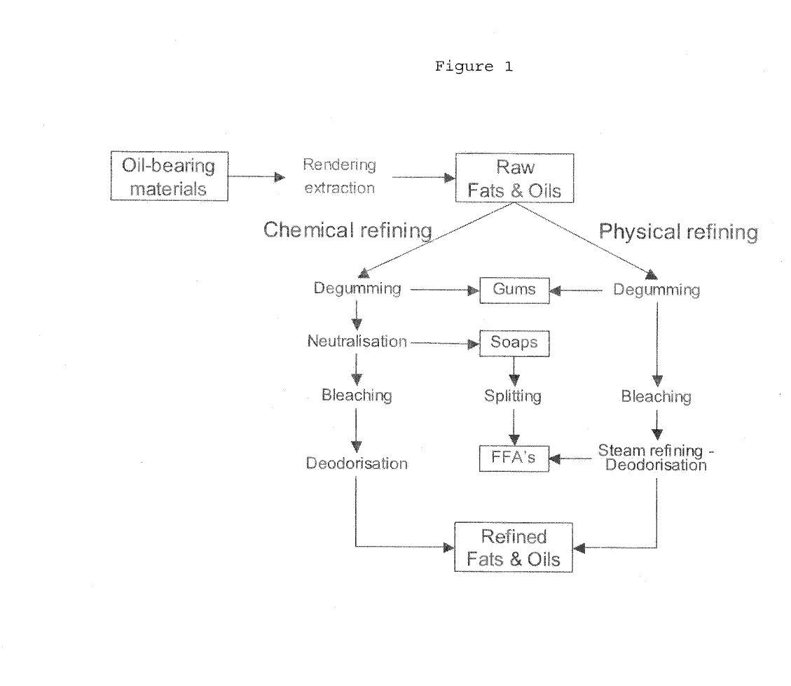

FIG. 1 illustrates the refining pretreatment in which crude oils are processed through various routes, physical or chemical, to Refined Bleached Deodorized (RBD) oils. Physical refining and alkali/chemical refining differ principally in the way free fatty acids are removed.

In chemical refining, FFA, most of the phosphatides, and other impurities are removed during neutralization with an alkaline solution, usually NaOH.

In physical refining, the FFA is removed by distillation during deodorisation and the phosphatides and other impurities must be removed prior to steam distillation.fats & oils

Currently, the refining method of choice is determined by the characteristics of the individual crude fats & oils:
(1) fats and oils that are normally physically refined;
(2) fats and oils that can be physically or chemically refined; and
(3) fats and oils that can only be chemically refined.

Table 2 below summarizes advantages and disadvantages of each treatment:

TABLE 2

| Refining type | Advantages | Disadvantages |
|---|---|---|
| Chemical | Functional process | Production of by- |
| refining | | products |
| | Not restricted by | Expensive process |
| | the oil type | |
| | Successful | High loss of oil |
| | reduction of FFA | |
| Physical | Cheaper | Not suitable for all |
| refining | | types of oils |
| | Less by-products | Requires high |
| | | temperature and vacuum |
| | Less energy | Can form undesired |
| | consumed | side reaction products |

Physical Refining

The physical refining can remove the FFA, as well as the unsaponifiables and other impurities by steam stripping, thus eliminating the production of soapstock and keeping neutral oil loss to a minimum. However, degumming pretreatments of the crude fats & oils are still required to remove those impurities that darken or otherwise cause a poor-quality product when heated to the temperature required for steam distillation. A degumming process is crucial for physical refining but optional for chemical refining. It consists of the treatment of crude oils, with water, salt solutions, enzymes, caustic soda, or diluted acids such as phosphoric, citric or maleic to remove phosphatides, waxes, pro-oxidants and other impurities. The degumming processes convert the phosphatides to hydrated gums, which are insoluble in oil and readily separated as a sludge by settling, filtering or centrifugal action. After degumming, phosphorous must be less than 30 ppm. So that bleaching or dry degumming can further reduce this level to less than 5 ppm and remove all traces of iron and copper. Acid or enzymatic degumming processes are normally employed to achieve these results.

The various industrial degumming processes have different aims. Fats & oils to be degummed vary widely in gum content and gum properties and finally, the means of gum disposal available, what equipment is needed and/or available, and the cost of auxiliaries also influence the choice of the most appropriated degumming process. The lipid handbook (The lipid handbook, edited by Frank D. Gunstone, John L. Harwood, Albert J. Dijkstra. 3rd ed., chapter 3.4) deals with these aspects in details. Next is briefly described the four major degumming process applied on the market.

The main purposes of the water degumming process are to produce oil that does not deposit a residue during transportation and storage, and to control the phosphorus content of crude oils just below 200 ppm. This process involves the addition of live steam to raw oil for a short period. The proper amount of water is normally about 75% of the phosphatides content of the oil. Too little water produces dark viscous gums and hazy oil, while too much water causes excess oil losses through hydrolysis. Water-degummed oil still contains phosphatides (between 80 and 200 ppm); only hydratable phosphatides are removed with this process. The nonhydratable phosphatides, which are calcium and magnesium salts of phosphatic acid and phosphatidyl ethanolamine, remain in the oil after water degumming.

Acid degumming process leads to a lower residual phosphorus content than water degumming and is therefore a good alternative if dry degumming and physical refining are to be the next refining steps. The acid degumming process might be considered as a variant of the water degumming process in that it uses a combination of water and acid. The non-hydratable phosphatides can be conditioned into hydratable forms with acid degumming. Phosphoric and citric acids are used because they are food grade, sufficiently strong and they bind divalent metal ions. Several acid degumming processes have been developed to attain a phosphorus value lower than 5 ppm that is required for good quality physically refined oils.

An acid refining differs from the acid degumming by the neutralisation of the liberated phosphatides (the action of the degumming acid does not lead to full hydration of the phosphatides) to make them hydratable by the addition of a base.

In dry degumming process, the oil is treated with an acid (principle is that strong acids displace weaker acids from their salts) to decompose the metal ion/phosphatides complex and is then mixed with bleaching earth. The earth containing the degumming acid, phosphatides, pigments and other impurities is then removed by filtration. Seed oils that have been water or acid-degummed may also be dry degummed to ensure a low phosphorus oil to steam distillation. An increase in FFA of less than 0.2% should be expected but the final phosphorus content must be reduced to less than 5 ppm. This process constitutes the main treatment for palm oil, lauric oils, canola oil and low phosphatides animal fats, such as tallow or lard. The dry degumming process allows crude oil to be fully refined in only two steps: dry degumming and physical refining.

In enzymatic degumming process, Phospholipase Al, the lastest developed degumming enzyme, changes the phospholipids into lysophospholipids and free fatty acids.

This process has three important steps:

(1) adjustement of the pH with a buffer;

(2) enzymatic reaction in the holding tanks; and

(3) separation of the sludge from the oil.

Oil to be degummed enzymatically by this way can be crude or water degummed.

The lipid handbook (The lipid handbook, edited by Frank D. Gunstone, John L. Harwood, Albert J. Dijkstra. 3rd ed.) describes many variants and details of the degumming processes.

The purpose of bleaching is to provide a decoloured oil but also to purify it in preparation for further processing. All fully refined oils have been subjected to one or the other bleaching process. Refined oil contains traces of a number of undesirable impurities either in solution or as colloidal suspensions. The bleaching process does more than just increasing the transmission of light through the oil and is often called “adsorptive cleaning.”. The bleaching process is often the first filtration encountered by the oil, so it ensures the removal of soaps, residual phosphatides, trace metals, and some oxidation products, and it catalyzes the decomposition of carotene and the adsorbent also catalyzes the decomposition of peroxides. These non-pigment materials, such as soap, gums, and pro-oxidants metals, which hinder filtration, poison hydrogenation catalyst, darken the oils, and affect finished oil flavour. Another function is the removal of the peroxides and secondary oxidation products. The key parameters for the bleaching process are procedure, adsorbent type and dosage, temperature, time, moisture and filtration, as shown in the Lipid Handbook (The lipid handbook, edited by Frank D. Gunstone, John L. Harwood, Albert J. Dijkstra. 3rd ed., chapter 3.7). The three most common types of contact bleaching methods used for edible fats and oils are batch atmospheric, batch vacuum and continuous vacuum. Chemical agents have been used or proposed for use but practically all edible oil decolouration and purification is accomplished with adsorptive clays, synthetic amorphous silica and activated carbons.

Before the last major processing step, bleached oil can be hydrogenated, for two reasons. One reason is to change naturally occurring fats & oils into physical forms with the consistency and handling characteristics required for functionality. The second reason for hydrogenation is to increase the oxidation and thermal stability. Instead of purification in other described processes, this step consists in fats & oils molecular modification.

Hydrogen is added directly to react with unsaturated oil in the presence of catalysts, mostly nickel. This process greatly influences the desired stability and properties of many edible oil products. The hydrogenation process is easily controlled and can be stopped at any point. A gradual increase in the melting point of fats and oils is one of the advantages. If the double bonds are eliminated entirely with hydrogenation, the product is a hard brittle solid at room temperature. Shortening and margarine are typical examples. A wide range of fats and oils products can be produced with the hydrogenation process depending upon the conditions used, the starting oils, and the degree of saturation or isomerization.

To obtain good-quality fats and oils with physical refining, it is essential to have a phosphorous content lower than 5 ppm before steam stripping.

The degummed-bleached oils are vacuum stripped. This process encompasses the deodorisation process, applied after the alkali routes, as well as physical refining. Deodorisation, the last major processing step during which the FFA can be removed, is a vacuum-steam distillation process (1-2 mbar of residual pressure) at elevated temperature (180-240° C.) during which FFAs and minute levels of odoriferous materials, mostly arising from oxidation, are removed to obtain a bland and odourless oil. In order to volatilise the undesired high-boiling components, a deep vacuum and dilution with steam is applied so that the boiling temperature can be minimized.

The deodorisation utilizes the differences in volatility between off-flavour and off-odor substances and the triglycerides.

The odoriferous substances, FFAs, aldehydes, ketones, peroxides, alcohols, and others organic compounds are concentrated in a deodorizer distillate. Efficient removal of these substances depends upon their vapour pressure, for a given constituent is a function of the temperature and increases with the temperature.

As usually the last stage in the refining process, deodorisation has an important effect an overall refined oil quality and distillate composition. Its main purposes are giving a bland taste and smell, low FFA content, high oxidative stability and light and stable colour. Because of the need of a rather high temperature to remove the undesired components, unwanted side effects are, isomerisation of double bond, polymerisation, intra-esterification and degradation of vitamins and anti-oxidants. New dry condensing (steam is condensed into ice) vacuum systems capable of reaching a very low operating pressure in the deodorizer were introduced (close to 0.1 kPa). This progress allows a reduction of the deodorisation temperature without affecting the stripping efficiency in a negative way. In order to minimise the time that the oil is at high temperature, deodorizers can operate at dual temperatures to reach the best compromise between required residence time for deodorizing (at moderate temperature) and heat bleaching and final stripping at high temperature.

Deodorizer distillate is the material collected from the steam distillation of edible oils. The distillate from physically refined oils consists mainly of FFAs with low levels of unsaponifiable components. The concentration of FFA can be improved from typical 80% up to 98% by applying double condensing system that produces an enriched FFA cut. The distillate can be used as a source of industrial fatty acids or mixed with the fuel oil used to fire the steam boilers.

A physical refining will be preferred due to higher remaining FFA content in refined oils before steam stripping.

Chemical Refining

As applied to crude oils, it includes degumming (removal of phospholipids), neutralization (removal of free fatty acids), bleaching (decolourisation) and deodorisation (FIG. 1).

Degumming involves for instance the addition of water to hydrate any gums present, followed by centrifugal separation. Non-hydratable gums are removed by converting them first to a hydratable form using phosphoric or citric acid, followed by the addition of water and centrifugation. Acid degumming can also be used (see the description above).

The following step is neutralization in which an aqueous alkali, typically caustic soda or sodium carbonate, is sprayed into the oil which has been preheated to around 75-95° C. The alkali reacts with free fatty acids in the oil to form soaps, which are separated by settling or centrifugation. Selection of the aqueous alkali strength, mixing time, mixing energy, temperature, and the quantity of excess caustic all have an important impact on making the chemical refining process operate efficiently and effectively. A drying step may be incorporated after neutralization to ensure the complete removal of the added water. The soap can be used as such or can be hydrolyzed (acidulation) with sulphuric acid into the corresponding FFA.

The neutralized oil is bleached to remove colouring matter (such as carotenoids) and other minor constituents, such as oxidative degradation products or traces of metals. Bleaching uses activated fuller's earth with treatments typically in the 90-130° C. range for 10-60 minutes. The earth is sucked into the oil under vacuum and is removed by filtration.

The bleached oil is steam distilled at low pressure to remove volatile impurities including undesirable odours and flavours. This process, known as deodorisation, takes place in the temperature range of 180-270° C. and may last 15 minutes to five hours depending upon the nature of the oil, the quantity, and the type of equipment used.

### Hydrolysis

Concerning hydrolysis processes for triglycerides, they are well known processes. Detailed information about fat & oil splitting has been published by Sonntag (Sonntag, N. O. V., J. Am. Oil. Chemists'. Soc., Vol. 56, November 1979, p. 729A-732A) and in the Kirk-Othmer encyclopedia of chemical technology 5th ed. vol 22 p.738-740. In addition, the Bailey's Industrial Oil and Fat Products (ed. F. Shahidi, 2005, John Wiley & Sons) is also a well known source of information concerning those processes. As matter of non limiting examples, the following process can be considered:
a) the Twitchell process developed in 1898, which involves atmospheric boiling of fat in the presence of various reagents. In particular, sulfuric acid and a catalyst (sulfonic acid derivates) are used to perform the reaction. The process consists of three or four successive reboilings with fresh water containing reagent (occasionally spent waters containing glycerol are used in place of the fresh water). The splitting efficiency is not better than 95%, steam consumption is important, but the equipment is cheap. A final water wash removes sulfuric and sulfonic acids otherwise those acids would lead to corrosion in the distillation equipments. Fatty acid obtained can further be purified via distillation.
b) medium pressure (10 to 35 bars) autoclave splitting with a catalyst, such as ZnO or calcium or magnesium oxides. By using high pressure, removal of glycerol is not required as it is in the Twintchell process. With one batch, efficiency up to 95-96% can be achieved with high water concentrations (30% wt of the weight of the fat employed). Usually, 2-4% of ZnO based on the weight of fat is used.
c) Low pressure splitting with catalyst consists in using superheated steam in the presence of a catalyst. With a ZnO catalyst hydrolysis starts at 200-280° C. Use of superheated steam allows to decrease the pressure.
d) continuous, high pressure countercurrent splitting also known as the Colgate-Emery process. The reaction is carried out under conditions where water possesses significant (10-25%wt) solubility in fats and oils. In practice the reaction is performed at pressure in the range of 40 to 55 bars and temperature in the range of 240-270° C. in a tubular reactor. The water ratio is in the range of 40-50% wt of fat. ZnO can optionally be added as a catalyst to facilitate the reaction. The fats and oils are added in the bottom of the column and the water at the top of the column. Water and oils are moving counter currently through the column. The hydrolysis reaction of the fats and oils liberates glycerol and fatty acids. The glycerol is carried in the aqueous phase in the bottom of the column while the fatty acids are recovered in the top of column. Efficiency up to 99% can be obtained with a residence time of about 90 min. The purity of the fatty acid can be improved by removing partially hydrolyzed triglycerides with a vacuum distillation step.
e) enzymatic fat splitting. It can be performed at low temperature (even room temperature) and atmospheric pressure. The enzymatic fat splitting using enzymes, so called lipases, as biocatalysts acting on a water/oil mixture is described in the following publications: “Continuous Use of Lipases in Fat Hydrolysis”, M. Biihler and Chr. Wandrey, Fat Science Technology 89/Dec. 87, pages 598 to 605; “Enzymatische Fettspaltung”, M. Buhler and Chr. Wandrey, Fat Science Technology 89/Nr.4/1987, pages 156 to 164; and “Oleochemicals by Biochemical Reactions?”, M. Biihler and Chr. Wandrey, Fat Science Technology 94/No. 3/1992, pages 82 to 94. By means of this splitting technique, the oil or fat, respectively, is split into glycerol and free fatty acids. The glycerol migrates into the water phase whereas the organic phase enriches more and more with free fatty acids until, finally, only the free fatty acids remain in the organic phase.

Concerning glycerol purification, there are numerous methods to purify glycerol (Ullmann's Encyclopedia of Industrial Chemistry Vol. 15 p681-682 6^{th} edition).

For instance, raw glycerol can be purified by vacuum distillation. However care must be taken not to deteriorate the glycerol. At temperature in the range of 170 to 180° C., glycerol degrades and can also polymerize and generate impurity. In order to obtain high purity glycerol, two distillations can be performed.

Other purification of for instance glycerol with high salts content includes ion exclusion chromatography. It consists in passing the glycerol over a cationic, strongly acidic exchange resin. The ionic compounds remain in the liquid volume between the resin particles (Donnan effects) whereas the non ionic components concentrate in the resin pores. The ionic compounds are then washed from the column during a second step via elution.

Glycerol can also be purified by thin-film distillation. Thin films of glycerol are produced by a rotor. They are spread on the inner wall of a column. This column is heated the glycerol vaporized whereas the residues flow down to the bottom of the column. The thermal stress seen by glycerol is reduced because the residence time is minimized in the column limiting therefore the degradation risks.

Glycerol purification can also be purified via ion exchange. This process allows removal of inorganic salts, fat and soap components, colored matter, odor causing substances and other impurities. Crude glycerol is passed over a cationic exchange resin and then over an anionic exchange resin. The first resin removes the cations and the second resin removes the anions. Charged impurities are thereby removed and ultimately exchanged for water.

Additional details about glycerol purification can be found in Bailey's Industrial Oil and Fat Products (ed. F. Shahidi, 2005, John Wiley & Sons).

Other processes have been developed. For example, the U.S. Pat. No. 4,655,879 described a process of very deep glycerol purification which implies a large number of stages in which raw glycerol is initially alkalized in the presence of air for oxidation, then distilled at high reduced temperatures under pressure. As the glycerol obtained has a non desired color, it is in addition necessary to carry out an additional treatment to the activated carbon.

The U.S. Pat. No. 4,990,695 described the purification of raw glycerol with a combination of operations such as the adjustment of the pH in a range of 9 to 12, heating of the medium with 100° C., microfiltration and then ultrafiltration. The glycerol obtained is then distilled, possibly after a treatment with ion exchanger compounds.

Fractionation Treatment into Phases L and S

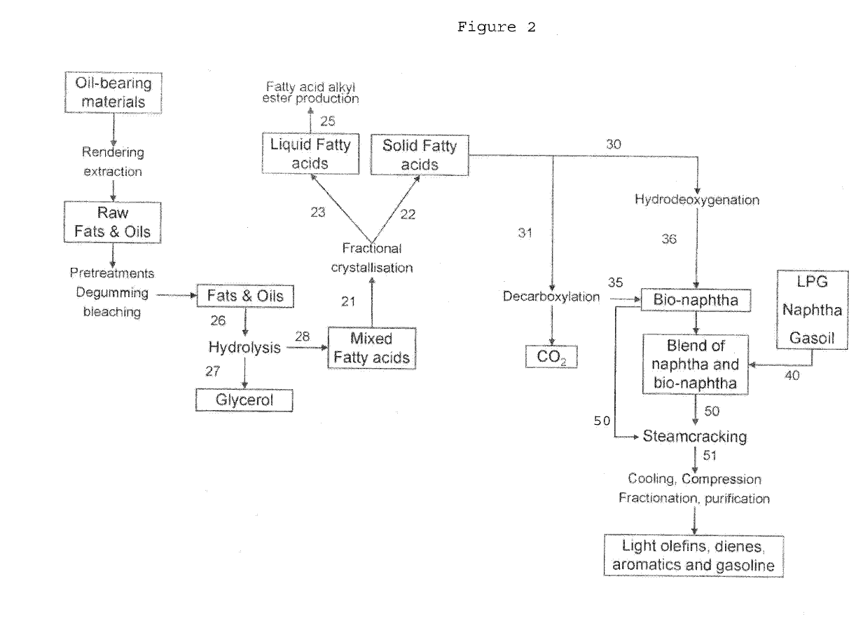

FIG. 2 illustrates where fatty acids are separated in a liquid fraction and a solid fraction, namely phases L and S, respectively.

The fractionation according to the present invention or “dry fractionation” or “dry winterization” is the removal of solids by controlled crystallization and separation techniques involving the use of solvents or dry processing (sometimes also referred to as dewaxing). It relies upon the difference in melting points to separate the oil fractions. The fractionation process has two main stages, the first being the crystallization stage. Crystals grow when the temperature of the molten fat & oil or its solution is lowered, and their solubility at the final or separation temperature determines the fatty acids composition of the crystals formed as well as their mother liquor. Separation process is the second step of fractionation. Several options have been reported, such as vacuum filters, centrifugal separators, conical screen-scroll centrifuges, hydraulic presses, membrane filter presses, or decanters with each their own advantages and drawbacks.

Fractionation can occur spontaneously during storage or transport, and this forms the basis of the dry fractionation process. This process is the oldest process type and thanks to steadily improved separation methods it has become competitive on product quality grounds with other, more expensive processes, such as solvent and detergent fractionation.

Fractionation can also been carried out in presence of solvents, like paraffins, alkyl-acetates, ethers, ketons, alcohols or chlorinated hydrocarbons. The use of solvents accelerates the crystallization and allows to crystallize more material before the slurry can no more be handled.

The term “fractional crystallization” will be used throughout this text and encompasses winterisation, dry fractionation and solvent fractionation.

Obtention of Bio-Diesel from Phase L

Methyl esters are produced from liquid fatty acids by esterification with alcohols.

Concerning the esterification of fatty acids, it is a common process used in the industry. For instance Bayer Technology Services commercializes the process BayFAME®. In this process, the free fatty acids (FFA) naturally present in triglycerides are esterified with methanol to produce fatty acid methyl esters (FAME). Once the FFA had been converted into FAME, the remaining triglycerides are sent to a classical bio diesel unit where they are transesterified. The overall process produces FAME that will be used as bio diesel. The esterification of FFA is catalyzed by a heterogeneous acid catalyst. The reaction is performed on acidic resin such as Amberlyst™ BD20. The process consists in a multi step process with inter-stage removal of by-products. The reaction being thermodynamically equilibrated, excess of methanol is required.

U.S. Pat. No. 4,652,56 describes similarly the preparation of fatty acid methyl esters (FAME) from triglycerides and free fatty acids (FFA) in two steps. In the first step, the FFA are esterified in presence of an excess of alcohol (methanol for instance). Preferably a molar ratio of about 25:1 (percent by volume of methanol over the volume of triglyceride starting material) is employed. A catalyst such as sulfuric acid and glycerol monosulfuric acid is required to perform the reaction. The catalyst content is in the range of 0.5 to 1.0 percent by weight of catalyst over the fat or oil starting material. The reaction is performed at temperature in the range of 65° C. (the boiling temperature) and pressure in the range of the atmospheric pressure. Once the reaction performed, the two phases (the methanol and organic phases) are separated. The process is then continued with the transesterification of remaining triglycerides.

EP2348009 discloses a method for preparing fatty acid alkyl ester for bio-diesel fuel, wherein fatty acid, specifically fatty acid distillate reacts with alcohol. This patent discloses a particular process design consisting of putting the alcohol stream and the fat and oil stream counter currently. A column with trays is used. On each tray of the column a temperature of 200 to 350° C. and a pressure of 1 to 35 bar are applied. The fatty acid is fed to an upper part and the alcohol is fed to a lower part of the counter current column reactor. A particular design of the column trays allows a good mixture of the methanol and of the fatty acids.

EP1424115 describes a packing-containing column with both reactor and distillation functions to perform fatty acid esterification. A catalyst is fixed to the packaging in the upper part of the column where the esterification reaction is performed. The column also separates the water and the alcohol on the top of the column and the ester in the bottom.

Obtention of Bio-Naphtha from Phase S

Two options exist to convert phase S fatty acids into LPG and naphtha-like hydrocarbons that can be used for the steamcracking in order to produce light olefins, dienes and aromatics. These are summarized in Table 3.

TABLE 3

| | | Catalyst/Intermediate |
|---|---|---|
| Feedstock | Process | compounds |
| Fatty acids | Catalytic | Supported Ni, Mo, |
| | Hydrodeoxygenation | Co, NiW, NiMo, CoMo, |
| | Catalytic | NiCoW, NiCoMo, NiMoW |
| | Decarboxylation | and CoMoW oxides or |
| | | sulphides |
| | | Supported group 10 |
| | | (Ni, Pt, Pd) or |
| | | group 11 (Cu, Ag) |
| | | metals or alloys |
| | | Basic oxides or |
| | | mixed basic oxides |
| Fatty acids | Thermal | Soaps of alkali, |
| Soaps | Decarboxylation | alkaline earth, |
| | | lanthanides or |
| | | group 12 or 13 |

The first option consists in decarboxylation or decarbonylation of fatty acids. These fatty acids can be obtained from fats & oils by physical refining (including steam/vacuum distillation), by (steam) splitting of triglycerides or by splitting of soaps (acidulation) using acids. Decarboxylation of carboxylic acids has been reported in 1982 (W.F. Maier, Chemische Berichte, 115, pages 808-812, 1982) over Pd/Si0₂ and Ni/Al₂O₂ catalysts in the gas phase. A highly selective decarboxylation has been reported in 2005 (I. Kubickova, Catalysis Today, 106, pages 197-200, 2005 and M. Snare, Industrial Engineering, Chemistry Research, 45, p. 5708-5715, 2006) using transition metal catalysts. Palladium based catalysts exhibit the highest selectivity towards decarboxylation. Carboxylic acids can also be decarboxylated under catalytic conditions using basic catalyst, like MgO, ZnO and mixed basic oxides (A. Zhang *, Q. Ma, K. Wang, X. Liu, P. Shuler, Y. Tang, “Naphthenic acid removal from crude oil through catalytic decarboxylation on magnesium oxide”, Applied Catalysis A: General 303, p. 103, 2006; A. More,

John R. Schlup, and Keith L. Hohn “Preliminary Investigations of the Catalytic Deoxygenation of Fatty Acids”, AIChe, The 2006 annual meeting, San Francisco and B. Kitiyanan, C. Ung-jinda, V. Meeyoo, “Catalytic deoxygenation of oleic acid over ceria-zirconia catalysts”, AIChe The 2008 annual meeting).

The following reactions can occur:

Decarboxylation:

R—CH₂—CH₂—COOH→R—CH₂—CH₃+CO₂

Decarbonylation:

R—CH₂—CH₂—COOH→R—CH═CH₂+CO+H₂O

Decarboxylation is preferentially done in presence of solid catalyst in batch type tank reactors, continuous fixed bed type reactors, continuous stirred tank reactors or slurry type reactors. The catalyst can be selected among Ni, Mo, Co or mixtures like NiW, NiMo, CoMo, NiCoW, NiCoMo, NiMoW and CoMoW oxides or sulphides as catalytic phase, preferably supported on high surface area carbon, alumina, silica, titania or zirconia or group 10 (Ni, Pt and Pd) and group 11 (Cu and Ag) metals or alloy mixtures supported on high surface area carbon, magnesia, zinc-oxide, spinels (Mg₂Al₂O₄, ZnAl₂O₄), perovskites (BaTiO₃, ZnTiO₂), calciumsilicates (like xonotlite), alumina, silica or silica-alumina's or mixtures of the latter. It is preferred that the support for the catalytic active phase exhibit low acidity, preferable neutral or basic in order to avoid hydro-isomerisation reactions that would result in branched paraffins and cracking. Decarboxylation can also be carried out on basic oxides, like alkaline oxides, alkaline earth oxides, lanthanide oxides, zinc-oxide, spinels (Mg₂Al₂O₄, ZnAl₂O₄), perovskites (BaTiO₂, ZnTiO₃), calciumsilicates (like xonotlite), either as bulk material or dispersed on neutral or basic carriers, on basic zeolites (like alkali or alkaline earth low silica/alumina zeolites obtained by exchange or impregnation).

Although, the decarboxylation reaction does not require hydrogen, it is preferred that the decarboxylation is done in presence of hydrogen that will stabilize the catalytic activity by removing strongly adsorbed unsaturated species (for instance when decarbonylation is the prevalent reaction pathway) from the catalyst surface by hydrogen-addition reactions. The presence of hydrogen can also hydrogenate the double bonds present in the acyl-moiety of the fatty acid in order to obtain paraffinic reaction products from the decarboxylation process. The decarboxylation of the fatty acids can be carried out at 100 to 550° C. in absence or presence of hydrogen at pressures ranging from 0.01 up to 10 MPa. The hydrogen to feedstock ratio is from 0 to 2000 N1/1.

Other reactions that can occur under the decarboxylation conditions are:

R—CH═CH₂+H₂→R—CH₂—CH₃

Hydrodeoxygenation of fatty acids:

R—CH₂—CH₂—COOH+3 H₂→R—CH₂—CH₂—CH₂ +2 H₂O

Further hydrogenation of the intermediate CO/CO₂ can occur depending on the amount of available hydrogen, the catalyst and the operating conditions:

CO +3 H₂→CH₄+H₂O

CO₂+4 H₂→CH₄+2 H₂O

A second option to obtain bio-naphtha from fats & oils is through the thermal decarboxylation of soaps of fatty acids. The soaps can be obtained from the chemical refining of fats & oils by neutralization, producing refined triglycerides and soaps, by neutralization of fatty acids obtained after (steam) splitting of fats & oils or by direct saponification of fats & oils using basic oxides or basic hydroxides, producing a soap and glycerol.

Decarboxylation has been carried out by decomposition of fatty acids in hot compressed water with the aid of alkali-hydroxides, resulting in the production of alkanes and CO₂ (M. Watanabe, Energy Conversion and Management, 47, p. 3344, 2006). Calcium-soaps of Tung oil have been reported to decompose by distillation as early as 1947 (C.C, Chang, S. W, Wan, “China's Motor Fuels from Tung Oil”, Ind. Eng. Chem, 39 (12), p. 1543, 1947; Hsu, H. L., Osburn, J. O., Grove, C. S., “Pyrolysis of the calcium salts of fatty acids”, Ind. Eng. Chem. 42 (10), p. 2141, 1950; Craveiro, A.A.; Matos, F. J. A.; Alencar, J.W.; Silveira E. R. Energia: Fontes Alternativas 3, p. 44, 1981; A. Demirbas, “Diesel fuel from vegetable oil via transesterification and soap pyrolysis”, Energy Sources 24 9, p. 835, 2002).

The preferred soaps are those made of alkaline, alkaline earth, lanthanide, zinc or aluminium cations. The thermal decarboxylation of soap can be carried out by heating until the molten soap starts to decompose into the corresponding paraffins or olefins and the corresponding metal-carbonate or metal-oxide/hydroxide and CO₂. Without willing to be bound to any theory, it is believed that the following overall reactions occur:

[R—CH₂—CH₂—COO⁻]ₓM^{x+}+x H₂O→x R—CH₂—CH₃+M[HCO₃]ₓ

M[HCO₃]ₓ←→M[OH]ₓ+CO₂

It is preferred that the thermal decomposition of the soaps is carried out in the presence of liquid, supercritical or vaporous water.

Steamcracking

Steamcrackers are complex industrial facilities that can be divided into three main zones, each of which has several types of equipment with very specific functions: (i) the hot zone including: pyrolysis or cracking furnaces, quench exchanger and quench ring, the columns of the hot separation train (ii) the compression zone including: a cracked gas compressor, purification and separation columns, dryers and (iii) the cold zone including: the cold box, de-methaniser, fractionating columns of the cold separation train, the C₂ and C₃ converters, the gasoline hydrostabilization reactor Hydrocarbon cracking is carried out in tubular reactors in direct-fired heaters (furnaces). Various tube sizes and configurations can be used, such as coiled tube, U-tube, or straight tube layouts. Tube diameters range from 1 to 4 inches. Each furnace consists of a convection zone in which the waste heat is recovered and a radiant zone in which pyrolysis takes place. The feedstock-steam mixture is preheated in the convection zone to about 530-650° C. or the feedstock is preheated in the convection section and subsequently mixed with dilution steam before it flows over to the radiant zone, where pyrolysis takes place at temperatures varying from 750 to 950° C. and residence times from 0.05 to 0.5 second, depending on the feedstock type and the cracking severity desired. In an advantageous embodiment the residence time is from 0.05 to 0.15 second. The steam/feedstock (the steam/[hydrocarbon feedstock]) weight ratio is between 0.2 and 1.0 kg/kg, preferentially between 0.3 and 0.5 kg/kg. In an advantageous embodiment the steam/feedstock weight ratio is between 0.2 and 0.45 and preferably between 0.3 and 0.4. For steamcracking furnaces, the severity can be modulated by: temperature, residence time, total pressure and partial pressure of hydrocarbons. In general the ethylene yield increases with the temperature while the yield of propylene decreases. At high temperatures, propylene is cracked and hence contributes to more ethylene yield. The increase in severity thus obtained leads to a moderate decrease in selectivity and a substantial decrease of the ratio C₃═/C₂═. So high severity operation favors ethylene, while low severity operation favors propylene production. The residence time of the feed in the coil and the temperature are to be considered together. Rate of coke formation will determine maximum acceptable severity. A lower operating pressure results in easier light olefins formation and reduced coke formation. The lowest pressure possible is accomplished by (i) maintaining the output pressure of the coils as close as possible to atmospheric pressure at the suction of the cracked gas compressor (ii) reducing the pressure of the hydrocarbons by dilution with steam (which has a substantial influence on slowing down coke formation). The steam/feed ratio must be maintained at a level sufficient to limit coke formation.

Effluent from the pyrolysis furnaces contains unreacted feedstock, desired olefins (mainly ethylene and propylene), hydrogen, methane, a mixture of C₄'s (primarily isobutylene and butadiene), pyrolysis gasoline (aromatics in the C₆ to C₈ range), ethane, propane, di-olefins (acetylene, methyl acetylene, propadiene), and heavier hydrocarbons that boil in the temperature range of fuel oil. This cracked gas is rapidly quenched to 338-510° C. to stop the pyrolysis reactions, minimize consecutive reactions and to recover the sensible heat in the gas by generating high-pressure steam in parallel transfer-line heat exchangers (TLE's). In gaseous feedstock based plants, the TLE-quenched gas stream flows forward to a direct water quench tower, where the gas is cooled further with recirculating cold water. In liquid feedstock based plants, a prefractionator precedes the water quench tower to condense and separate the fuel oil fraction from the cracked gas. In both types of plants, the major portions of the dilution steam and heavy gasoline in the cracked gas are condensed in the water quench tower at 35-40° C. The water-quench gas is subsequently compressed to about 25-35 Bars in 4 or 5 stages. Between compression stages, the condensed water and light gasoline are removed, and the cracked gas is washed with a caustic solution or with a regenerative amine solution, followed by a caustic solution, to remove acid gases (CO₂, H₂S and SO₂). The compressed cracked gas is dried with a desiccant and cooled with propylene and ethylene refrigerants to cryogenic temperatures for the subsequent product fractionation: Front-end demethanization, Front-end depropanization or Front-end deethanization.

In a front-end demethanization configuration, tail gases (CO, H₂, and CH₄) are separated from the C₂+components first by de-methanization column at about 30 bars. The bottom product flows to the de-ethanization, of which the overhead product is treated in the acetylene hydrogenation unit and further fractionated in the C₂ splitting column. The bottom product of the de-ethanization goes to the de-propanization, of which the overhead product is treated in the methyl acetylene/propadiene hydrogenation unit and further fractionated in the C₃ splitting column. The bottom product of the de-propaniser goes to the de-butanization where the C₄'s are separated from the pyrolysis gasoline fraction. In this separation sequence, the H₂ required for hydrogenation is externally added to C₂ and C₃ streams. The required H₂ is typically recovered from the tail gas by methanation of the residual CO and eventually further concentrated in a pressure swing adsorption unit.

Front-end de-propanization configuration is used typically in steamcrackers based on gaseous feedstock. In this configuration, after removing the acid gases at the end of the third compression stage, the C₃ and lighter components are separated from the C₄₊ by de-propanization. The de-propanizer C₃− overhead is compressed by a fourth stage to about 30-35 bars. The acetylenes and/or dienes in the C₃− cut are catalytically hydrogenated with H₂ still present in the stream. Following hydrogenation, the light gas stream is de-methanized, de-ethanized and C₂ split. The bottom product of the de-ethanization can eventually be C₃ split. In an alternative configuration, the C₃− overhead is first de-ethanised and the C₂− treated as described above while the C₃'s are treated in the C₃ acetylene/diene hydrogenation unit and C₃ split. The C₄+ de-propanizer bottom is de-butanized to separate C₄'s from pyrolysis gasoline.

There are two versions of the front-end de-ethanization separation sequence. The product separation sequence is identical to the front-end de-methanization and front-end depropanization separation sequence to the third compression stage. The gas is de-ethanized first at about 27 bars to separate C₂− components from C₃+ components. The overhead C₂− stream flows to a catalytic hydrogenation unit, where acetylene in the stream is selectively hydrogenated. The hydrogenated stream is chilled to cryogenic temperatures and de-methanized at low pressure of about 9-10 bars to strip off tail gases. The C₂ bottom stream is split to produce an overhead ethylene product and an ethane bottom stream for recycle. In parallel, the C₃+ bottom stream from the front-end de-ethanizer undergoes further product separation in a de-propaniser, of which the overhead product is treated in the methyl acetylene/propadiene hydrogenation unit and further fractionated in the C₃ splitting column. The bottom product of the de-propaniser goes to the de-butanization where the C₄'s are separated from the pyrolysis gasoline fraction. In the more recent version of the front-end de-ethanization separation configuration, the cracked gas is caustic washed after three compression stages, pre-chilled and is then de-ethanized at about 16-18 bars top pressure. The net overhead stream (C₂−) is compressed further in the next stage to about 35-37 bars before it passes to a catalytic converter to hydrogenate acetylene, with hydrogen still contained in the stream. Following hydrogenation, the stream is chilled and de-methanized to strip off the tail gases from the C₂ bottom stream. The C₂'s are split in a low pressure column operating at 9-10 bars pressure, instead of 19-24 bars customarily employed in high pressure C₂ splitters that use a propylene refrigerant to condense reflux for the column. For the low-pressure C₂ splitter separation scheme, the overhead cooling and compression system is integrated into a heat-pump, open-cycle ethylene refrigeration circuit. The ethylene product becomes a purged stream of the ethylene refrigeration recirculation system.

The ethane bottom product of the C₂ splitter is recycled back to steam cracking. Propane may also be re-cracked, depending on its market value. Recycle steam cracking is accomplished in two or more dedicated pyrolysis furnaces to assure that the plant continues operating while one of the recycle furnaces is being decoked.

Many other variations exist of the above-described configurations, in particular in the way the undesired acetylene/dienes are removed from the ethylene and propylene cuts.

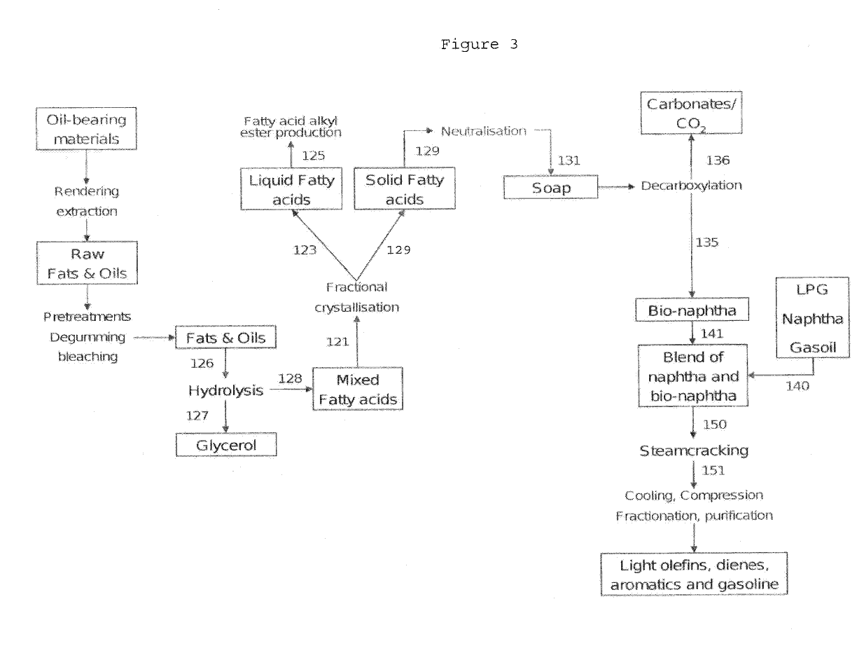

The different embodiments are represented in FIGS. 2 and 3.

In a first embodiment (FIG. 2), Fats & Oils 26 are hydrolyzed to recover mixed fatty acids 28 and glycerol 27. The mixed free fatty acids 27 are fractional crystallized 21, resulting in a phase S 22 and a phase L 23 fraction. The phase S 22 can be sent to a hydrodeoxygenation section 30 or to a decarboxylation section 31 where they are converted into bio-naphtha 35, 36. This bio-naphtha is sent direclty to the steamcracking 50 or blended with fossil LPG, naphtha or gasoil 40 and hence the blend is streamcracked 50. The products of the steamcracking are cooled, compressed, fractionated and purified 51. This results in light olefins (ethylene, propylene and butenes), dienes (butadiene, isoprene, (di)cyclopentadiene and piperylenes), aromatics (benzene, toluene and mixed xylenes) and gasoline as main components. The phase L 23, obtained from the fractional crystallization is sent to biodiesel production section 25.

In a second embodiment (FIG. 3), Fats & Oils 126 are hydrolyzed to recover mixed fatty acids 128 and glycerol 127. The mixed free fatty acids 127 are fractional crystallized 121, resulting in a phase S 129 and a phase L 123 fraction. The free fatty acids of phase S 129 are neutralized to produce soaps 131. The soaps can be sent to the decarboxylation section where they are converted into bio-naphtha 135 and metal-carbonates or CO₂ 136. This bio-naphtha is sent 141 to the steamcracking 150 or blended with fossil LPG, naphtha or gasoil 140 and hence the blend is streamcracked 150. The products of the steamcracking are cooled, compressed, fractionated and purified 151. This results in light olefins (ethylene, propylene and butenes), dienes (butadiene, isoprene, (di)cyclopentadiene and piperylenes), aromatics (benzene, toluene and mixed xylenes) and gasoline as main components. The phase L 123, obtained from the fractional crystallization is sent to biodiesel production section 125.

### EXAMPLES

### Exemple 1

Hydrodeoxygenation of a fatty acid feed has been evaluated under the following conditions:

In an isothermal reactor, 50 ml of a hydrotreating catalyst composed of Molybdenum and Nickel supported on alumina (prepared according to patent U.S. Pat. No. 6,280,610B1) was loaded, the catalyst dried and pre-sulfurised under standard conditions with straightrun gasoil with a initial boiling point of 187° C. and a final boiling point of 376° C. (a straight run gasoil is a gasoil cut obtained directly after distillation without any other treatment). This gasoil was doped with dimethyl di-sulphur (DMDS). The hydrodeoxygenation of fatty acid is done at:

LHSV=1 h⁻¹

Inlet Temperature=320° C.

Outlet pressure=60 bars

H2/oil ratio=1050 N1/1

Feedstock=oleic feed doped with 2.5 wt % DMDS

Table 4 shows a typical composition of the oleic feed.

The gas and liquid effluent are separated by means of a separator (gas/liquid) at atmospheric pressure. Gases are sent to a p-GC analyzer and liquids are sent to a sampler. The mass balance is around 101% and all product weights are calculated for 100g of treated feed.

TABLE 4
Typical composition of oleic feed

| Component | Wt. % |
|---|---|
| C14:0 | 1.6 |
| C16:0 | 8.2 |
| C16:1 | 0.4 |
| C18:0 | 3.8 |
| C18:1 | 64.7 |
| C18:2 | 20.6 |
| C18:3 | 0.1 |
| C20:0 | 0.3 |
| C20:1 | 0.3 |

The total liquid effluent is biphasic and need a separation step. The organic phase was analyzed via GC-MS. A complete analysis is reported in Table 5.

The liquid effluent is composed of 86.74 wt % of n-paraffins but it is composed of 99.99 wt % of interesting components, which could be sent to the naphtha-cracker.

TABLE 5
Material balance and complete GC analysis of hydrocarbon phase

| Feed | Products |
|---|---|
| 10.7 gr hydrogen | 8.87 gr CO₂ |
| 100 gr oleic feed (including DMDS) | 2.60 gr CO |
| | 8.50 H2 |
| | 0.00 gr propane |
| | 1.14 gr methane |
| | 5.65 gr water phase |
| | 85.18 gr hydrocarbon phase |
| Hydrocarbon phase composition | Wt % |
| n-paraffins with C₃ to C₁₄ | 2.403 |
| other paraffins with C₅ to C₁₄ | 0.221 |
| other C15 | 0.104 |
| n-C15 | 2.404 |
| other C16 | 0.337 |
| n-C16 | 1.384 |
| other C17 | 6.337 |
| n-C17 | 55.242 |
| other C18 | 3.662 |
| n-C18 | 23.417 |
| n-paraffins with C₁₉ to C₃₅ | 1.892 |
| other paraffins with C₁₉ to C₃₅ | 2.584 |
| Other oxygenates | 0.014 |
| Total | 100.00 |

86.74 wt % of the hydrocarbon phase are comprised of n-paraffins that is high quality bio-naphtha feedstock for a steamcracker. About 0.014 wt % of remaining oxygenates are found in the hydrocarbon phase. That corresponds to 15.7 wppm 0-atoms. Considering the 0 content in the oleic feed, that represents 11.33 wt % (or 113280 wppm 0-atoms), resulting in a hydrodeoxygenation conversion of 99.99%.

### Example 2

Hydrodeoxygenation of a triglyceride feed has been evaluated under the following conditions:

In an isothermal reactor, 10 ml of a hydrotreating catalyst composed of Molybdenum and Nickel supported on alumina (KF848 obtained from Albemarle) was loaded, the catalyst dried and pre-sulfurised under standard conditions with straightrun gasoil doped with DMDS. The hydrodeoxygenation of rapeseed is done at:

LHSV=1 h⁻¹

Inlet Temperature=320° C.

Outlet pressure=60 bars

H2/oil ratio=630 N1/1

Feedstock=rapeseed doped with 1 wt % DMDS

Table 6 shows a typical composition of the rapeseed oil.

The gas and liquid effluent are separated by means of a separator (gas/liquid) at atmospheric pressure. Gases are sent to a p-GC analyzer and liquids are sent to a sampler. The mass balance is around 99% and all product weights are calculated for 100g of treated feed.

TABLE 6
Typical composition of rapeseed oil

| Components | wt % |
|---|---|
| tetradecanoate | 0.1 |
| hexadecenoate | 0.2 |
| hexadecanoate | 4.8 |
| heptadecanoate | 0.1 |
| octadecadienoate | 20.6 |
| octadecenoate | 61.3 |
| octadécatrienoate | 8.6 |
| octadecanoate | 1.8 |
| eisosenoate | 1.2 |
| eicosanoate | 0.7 |
| docosenoate | 0.3 |
| docosanoate | 0.3 |
| | 100 |

The total liquid effluent is biphasic and need a separation step. The organic phase was analyzed via GC-MS. A complete analysis is reported in Table 7.

The liquid effluent is composed of 94.4 wt % of n-paraffins but it is composed of 99.94 wt % of interesting components, which could be sent to the naphtha-cracker.

TABLE 7
Material balance and complete GC analysis of hydrocarbon phase

| Feed | Products |
|---|---|
| 5.96 gr hydrogen | 6.48 gr CO₂ |
| 100 gr rapeseed | 0.55 gr CO |
| | 3.52 H2 |
| | 5.98 gr propane |
| | 0.18 gr methane |
| | 2.77 gr water phase |
| | 85 gr hydrocarbon phase |
| Hydrocarbon phase composition | Wt % |
| C3 | 0.005 |
| n-paraffins with C₅ to C₁₄ | 0.268 |
| other paraffins with C₅ to C₁₄ | 0.238 |
| other C15 | 0.061 |
| n-C15 | 2.353 |
| other C16 | 0.100 |
| n-C16 | 2.754 |
| other C17 | 1.633 |
| n-C17 | 41.077 |
| other C18 | 2.108 |
| n-C18 | 44.344 |
| dodecyl-cyclohexane | 0.168 |
| tridecyl-cyclopentane | 0.110 |
| n-paraffins with C₁₉ to C₃₅ | 3.599 |
| other paraffins with C₁₉ to C₃₅ | 1.1 |
| >n-C35 | 0.013 |
| 2-butanone | 0.034 |
| Other oxygenates | 0.025 |
| Total | 100.00 |

94.4 wt % of the hydrocarbon phase are comprised of n-paraffins that is high quality bio-naphtha feedstock for a steamcracker. About 0.059 wt % of remaining oxygenates are found in the hydrocarbon phase. That corresponds to 112 wppm 0-atoms. Considering the 0 content in the triglyceride feed, that represents 10.86 wt % (or 108600 wppm 0-atoms), resulting in a hydrodeoxygenation conversion of 99.89%.

### Example 3

n-Paraffins and conventional naphtha have been steamcracked under different severity conditions. Table 8 gives the results. It is evident from the results that such-obtained bio-naphtha are better feedstock for steamcracking compared to fossil naphtha.

Significant higher ethylene and propylene yields can be obtained whereas the methane make and the pyrolysis gasoline make is reduced with at least about 20%. The ultimate yield of HVC (High value Chemicals=H2+ethylene+propylene+butadiene+benzene) is above 70 wt %. Ethylene/Methane weight ratio is always above 3.

TABLE 8

| | Naphtha | n-Decane | n-C15 | n-C20 |
|---|---|---|---|---|
| P/E | 0.59 | 0.44 | 0.50 | 0.49 |
| COT | 812 | 812 | 812 | 812 |
| S/HC | 0.35 | 0.35 | 0.35 | 0.35 |
| Summary | wt % (dry) | wt % (dry) | wt % (dry) | wt % (dry) |
| Hydrogen | 0.87 | 0.66 | 0.59 | 0.57 |
| Methane | 14.79 | 11.67 | 10.65 | 10.00 |
| Acetylene | 0.25 | 0.25 | 0.25 | 0.25 |
| Ethylene | 25.39 | 38.87 | 36.24 | 35.82 |
| Ethane | 4.09 | 6.58 | 6.07 | 5.84 |
| Methyl-Acetylene | 0.29 | 0.21 | 0.22 | 0.22 |
| Propadiene | 0.21 | 0.15 | 0.16 | 0.16 |
| Propylene | 15.10 | 17.29 | 18.08 | 17.63 |
| Propane | 0.51 | 0.73 | 0.69 | 0.66 |
| Vinyl-Acetylene | 0.04 | 0.04 | 0.04 | 0.04 |
| Butadiene | 4.61 | 5.96 | 6.88 | 7.30 |
| Butene (sum) | 4.86 | 2.99 | 3.34 | 3.43 |
| Butane (sum) | 0.08 | 0.14 | 0.12 | 0.12 |
| Total C5-C9's | 23.69 | 12.48 | 14.65 | 15.75 |
| Total C10+ | 5.17 | 1.93 | 1.96 | 2.15 |
| Carbon Oxide | 0.05 | 0.05 | 0.05 | 0.05 |
| Carbon Dioxide | 0.00 | 0.00 | 0.00 | 0.00 |
| Ultimate Ethylene | 28.67 | 44.14 | 41.09 | 40.49 |
| C2= + C3= | 43.77 | 61.43 | 59.17 | 58.12 |
| BENZENE | 8.27 | 5.35 | 6.46 | 7.05 |
| HVC's | 54.25 | 68.14 | 68.24 | 68.37 |
| Ultimate HVC's | 57.52 | 73.40 | 73.10 | 73.04 |
| | Naphtha | n-Decane | n-C15 | n-C20 |
| P/E | 0.50 | 0.39 | 0.44 | 0.44 |
| COT | 832 | 832 | 832 | 832 |
| S/HC | 0.35 | 0.35 | 0.35 | 0.35 |
| Summary | wt % (dry) | wt % (dry) | wt % (dry) | wt % (dry) |
| Hydrogen | 0.96 | 0.76 | 0.69 | 0.67 |
| Methane | 16.25 | 12.80 | 11.80 | 11.15 |
| Acetylene | 0.36 | 0.37 | 0.37 | 0.37 |
| Ethylene | 26.91 | 39.67 | 36.93 | 36.47 |
| Ethane | 3.89 | 6.10 | 5.62 | 5.42 |
| Methyl-Acetylene | 0.36 | 0.26 | 0.27 | 0.27 |
| Propadiene | 0.25 | 0.18 | 0.19 | 0.19 |
| Propylene | 13.48 | 15.59 | 16.28 | 15.91 |
| Propane | 0.44 | 0.62 | 0.59 | 0.57 |
| Vinyl-Acetylene | 0.05 | 0.06 | 0.07 | 0.07 |
| Butadiene | 4.41 | 5.79 | 6.49 | 6.79 |
| Butene (sum) | 3.67 | 2.12 | 2.34 | 2.38 |
| Butane (sum) | 0.06 | 0.11 | 0.09 | 0.09 |
| Total C5-C9's | 22.30 | 13.14 | 15.33 | 16.42 |
| Total C10+ | 6.53 | 2.38 | 2.86 | 3.18 |
| Carbon Oxide | 0.07 | 0.07 | 0.07 | 0.07 |
| Carbon Dioxide | 0.01 | 0.00 | 0.00 | 0.00 |
| Ultimate Ethylene | 30.02 | 44.55 | 41.43 | 40.80 |
| C2= + C3= | 43.51 | 60.14 | 57.71 | 56.70 |
| BENZENE | 9.42 | 6.55 | 7.77 | 8.39 |
| HVC's | 55.18 | 68.35 | 68.16 | 68.23 |
| Ultimate HVC's | 58.29 | 73.23 | 72.66 | 72.56 |

| | |
|---|---|
| Naphtha composition | wt % |
| Normal paraffins | 31.26 |
| Iso paraffins | 33.48 |
| Naphtenics | 28.1 |
| Aromatics | 7.16 |
| Olefins | 0 |
| Others | 0 |

P/E is the propylene /ethylene ratio

COT is the coil outlet temperature

S/HC is the ratio steam/hydrocarbon

1-13. (canceled)

14. A process comprising:
optionally subjecting a complex mixture of natural occurring fats & oils to a refining treatment for removing a major part of non-triglyceride and non-fatty acid components, thereby obtaining refined oils;
subjecting the complex mixture or the refined oils to a hydrolysis step for obtaining glycerol and a mixture of free fatty acids;
subjecting the mixture of free fatty acids to a fractionation step by fractional crystallization for obtaining:
a liquid or substantially liquid free fatty acids part (phase L); and
a solid or substantially solid free fatty acids part (phase S);
transforming the phase L into alkyl-esters as bio-diesel by an esterification;
transforming the phase S into linear or substantially linear paraffins as bio-naphtha by:
hydrodeoxygenation or decarboxylation of the free fatty acids; or
obtaining fatty acids soaps from the phase S and transforming the fatty acids soaps into linear or substantially linear paraffins as the bio-naphtha by decarboxylation of the fatty acids soaps.

15. The process according to claim 14, wherein the complex mixture of natural occurring fats & oils is selected among vegetable oils and animal fats.

16. The process according to claim 14, wherein the complex mixture of natural occurring fats & oils is selected among inedible highly saturated oils, waste food oils, by-products of the refining of vegetable oils, or mixtures thereof.

17. The process according to claim 14, wherein the mixture of free fatty acids is fractioned into the phase L and the phase S by a fractional crystallization method comprising a controlled cooling down during which the free fatty acids of the mixture with saturated or substantially saturated acyl-moieties crystallize and precipitate from the mixture forming the phase S, while the free fatty acids with unsaturated or substantially unsaturated acyl-moieties remain liquid forming the phase L, wherein phase S and phase L are then separated by simple filtration, decantation, or centrifugation.

18. The process according to claim 14, wherein the phase L is esterified with a C1-C5 monofunctional alcohol in order to produce alkyl fatty esters as bio-diesel.

19. The process according to claim 14, wherein the fatty acids soaps are obtained by neutralization of free fatty acids obtained from hydrolysis of the complex mixture of natural occurring fats & oils.

20. The process according to claim 14, wherein the phase S is transformed into linear or substantially linear paraffins as bio-naphtha by hydrodeoxygenation, decarboxylation, or decarbonylation of the free fatty acids;
wherein the hydrodeoxygenation, decarboxylation, or decarbonylation is conducted in the presence of hydrogen and of at least one catalyst selected among Ni, Mo, Co, or mixtures thereof; or sulphides; or Group 10 and Group 11 metals or alloy mixtures thereof supported on high surface area carbon, magnesia, zinc-oxide, spinels, perovskites, calciumsilicates, alumina, silica or silica-alumina.

21. The process according to claim 14, wherein the phase S is transformed into linear or substantially linear paraffins as bio-naphtha by decarboxylation of the free fatty acids on basic oxides either as bulk material or dispersed on neutral or basic carriers.

22. The process according to claim 14, wherein the phase S is transformed into linear or substantially linear paraffins as bio-naphtha by decarboxylation of the free fatty acids on basic zeolites.

23. The process according to claim 14, wherein the phase S is transformed into linear or substantially linear paraffins as bio-naphtha by hydrodeoxygenation the free fatty acids; and
wherein the hydrodeoxygenation is carried out at a temperature from 200 to 500° C., under a pressure of from 1 MPa to 10 MPa, and with a hydrogen to feedstock ratio from 100 to 2000 N1/1.

24. The process according to claim 14, wherein the phase S is transformed into linear or substantially linear paraffins as bio-naphtha by decarboxylation of the free fatty acids; and
wherein the decarboxylation is carried out at a temperature from 100 to 550° C. in absence or presence of hydrogen under a pressure from 0.01 MPa to 10 MPa.

25. The process according to claim 14, wherein the phase S is transformed into linear or substantially linear paraffins as bio-naphtha by obtaining fatty acids soaps from the phase S and transforming the fatty acids soaps into linear or substantially linear paraffins as the bio-naphtha by decarboxylation of the fatty acids soaps; and
wherein the decarboxylation of the fatty acids soaps is carried out at a temperature of from 100 to 550° C., under a pressure of from 0.1 MPa to 10 MPa, and in the presence of water.

26. The process according to claim 25, wherein the decarboxylation of the fatty acids soaps is carried out with a water to feedstock ratio of at least 1 mole water per mole of fatty acids soap.

27. The process of claim 14, further comprising using the bio-naphtha as a direct feedstock of a steamcracker in order to obtain cracked products.

28. The process of claim 27, wherein the bio-naphtha is blended with at least a conventional feedstock selected among LPG, naphtha, and gasoil.

29. The process of claim 27, wherein the cracked products include bio-ethylene, bio-propylene, bio-butadiene, bio-isoprene, bio-(di)cyclopentadiene and bio-piperylenes, bio-benzene, bio-toluene, bio-xylene and bio-gasoline.

30. The process of claim 27, wherein the direct feedstock is mixed with steam in a ratio of 0.2 to 1.0 kg steam per kg direct feedstock, and wherein the mixture of the direct feedstock and steam is heated up to a temperature of from 750 to 950° C. at a residence time of 0.05 to 0.5 seconds.

31. The process of claim 30, wherein the direct feedstock is mixed with steam in a ratio of 0.3 to 0.5 kg steam per kg feedstock.

32. The process of claim 27, wherein the cracked products have an ethylene to methane weight ratio of at least 3.

33. The process of claim 14, wherein the complex mixture of natural occurring fats & oils are subjected to the refining treatment for removing a major part of non-triglyceride and non-fatty acid components, thereby obtaining the refined oils.
